(19) United States
(12) Patent Application Publication
(10) Pub. No.: US 2026/0191174 A1
(43) Pub. Date: Jul. 9, 2026

(54) METHOD FOR CONSTRUCTING PERIODONTITIS RAT MODEL AND APPLICATION THEREOF

(71) Applicant: School and Hospital of Stomatology, Guangzhou Medical University, Guangzhou (CN)

(72) Inventors: Miao Yu, Guangzhou (CN); Qianzhou Jiang, Guangzhou (CN); Henglong Cao, Guangzhou (CN); Ying Wu, Guangzhou (CN); Nannan Chen, Guangzhou (CN); Jianfeng Lin, Guangzhou (CN); Wanyi Wei, Guangzhou (CN); Jianzhao Luo, Guangzhou (CN); Yingyu Ma, Guangzhou (CN); Cheng Zhang, Guangzhou (CN); Ziyang Zhou, Guangzhou (CN)

(21) Appl. No.: 19/364,625

(22) Filed: Oct. 21, 2025

Related U.S. Application Data
(63) Continuation of application No. PCT/CN2025/123846, filed on Sep. 25, 2025.

(30) Foreign Application Priority Data
Jan. 3, 2025 (CN) 2025100057031

(51) Int. Cl. A01K 67/027

(52) U.S. Cl. CPC A01K 67/027; A01K 2227/105; A01K 2267/03

(57) ABSTRACT

A method for constructing a periodontitis rat model and its application is provided. The method includes the following steps: rats are infected with Toxoplasma gondii, followed by co-infection with Porphyromonas gingivalis and Treponema denticola in Toxoplasma gondii-infected rats to construct the periodontitis rat model. The rapid and stable acquisition of the periodontitis animal model is achieved through the synergistic effect between Porphyromonas gingivalis, Treponema denticola, and Toxoplasma gondii, and the constructed periodontitis rat model is highly consistent with clinical symptoms and signs of human periodontitis, reflecting the systemic nature and severity of periodontitis. In addition, this method has the advantages of rapid modeling, high success rate, and good reproducibility, making it convenient for studying the infection, development mechanisms, and prevention and treatment processes of periodontitis.

### CROSS-REFERENCE TO RELATED APPLICATION

This application claims priority to Chinese Patent Application No. 202510005703.1, filed on Jan. 3, 2025. The entire contents of the above-mentioned application are incorporated herein by reference.

### TECHNICAL FIELD

The disclosure relates to the technical field of periodontology, particularly to an animal model construction method used for studying pathogenesis of periodontitis and its associated infectious diseases, and more particularly to a method for constructing a periodontitis rat model and an application thereof in research on periodontitis-related diseases.

### BACKGROUND

Chronic periodontitis (CP) is a chronic infectious disease and the primary cause of tooth loss in adults, with a high prevalence worldwide. According to the fourth National Oral Health Epidemiological Survey data of China, the detection rate of dental calculus in Chinese adults aged 35-44 is 96.7%, the detection rate of gingival bleeding reaches 87.4%, an increase of 10.1% compared to 77.3% ten years ago, and the prevalence and severity increase with age.

Porphyromonas gingivalis (P. gingivalis) is the main pathogenic bacterium of the CP, possessing the ability to activate host inflammation and regulate immune responses. Treponema denticola (T. denticola), as an important bridge bacterium, can not only bind and colonize a tooth surface with early colonizing bacteria such as Streptococcus and Actinomycetes but also co-aggregate with subsequent bacteria such as P. gingivalis, Tannerella forsythia (T. forsythia), and Fusobacterium nucleatum (F. nucleatum), promoting the formation of plaque biofilm. At the same time, T. denticola has many virulence factors and directly participates in the destruction of periodontal tissues. Studies have shown that P. gingivalis may lead to the occurrence and development of the CP. The gingival bleeding is a typical clinical symptom of the CP, which provides convenient conditions for orally transmitted pathogens. Toxoplasma gondii (T. gondii) is an important foodborne parasitic protozoan. After infecting the body, T. gondii can flow with the blood to reach various parts of the body, damaging the brain, heart, etc., leading to a decline in human immunity.

In the related art, studies involving construction of periodontitis models have been conducted. For example, in the thesis “Experimental Study on the Immunoregulatory Role of Th1/Th2 Cell Response on Mouse Periodontitis” (China National Knowledge Infrastructure: Xie, Min, Experimental study on the immunoregulatory role of Th1/Th2 cell response in periodontitis of mice, Doctoral dissertation, Jinan University, 2008.), a mouse periodontitis animal model is constructed by a method of silk suture ligature and local bacterial coating, and further infection with ultraviolet-attenuated T. gondii is performed to explore a relationship between the body's immune response and periodontitis. However, this method uses bacteria from periodontitis patients for infection, and the proportion and concentration of bacterial species in dental calculus from different periodontitis patients vary, so infection using bacteria derived from the periodontitis patients cannot be standardized to meet the requirements of high success rate and high stability in model construction. In addition, although many periodontitis pathogenic bacteria are known in the related art, the properties of different bacteria vary greatly, and their effects on the same host also interact with each other, so the expected outcome after combined infection is poor.

Rats, as widely used rodent model animals in the study of disease pathogenesis, drug metabolism, and pharmacokinetics, have significant advantages over mice, such as larger size for easier operation, more blood volume with stronger tolerance, and larger available sample size. Given that the effective prevention and treatment of periodontitis have become a focus of attention for scholars both domestically and internationally, the efficient and rapid construction of a periodontitis rat model is of great significance for the research and prevention and treatment of periodontitis diseases. Based on the study of the pathogenesis of periodontitis, the disclosure unexpectedly finds that co-infection of rats with P. gingivalis, T. denticola, and T. gondii can rapidly and stably obtain a periodontitis rat animal model, which is easy to operate and has a high success rate.

### SUMMARY

A purpose of the disclosure is to provide a method for constructing a periodontitis rat model, aiming to rapidly and with high success rate obtain an animal model of periodontitis for research and application.

To achieve the above purpose, technical solutions adopted by the disclosure are as follows.
1. Construction of a T. gondii infection model: Each rat is administered 10 T. gondii cysts via oral gavage to construct the T. gondii infection model.
2. Construction of a periodontitis model: Periodontitis is simulated by ligating first maxillary molars with 3-0 silk suture and applying P. gingivalis and T. denticola intraorally.

Specific steps include rat anesthesia, tooth localization, silk suture ligature, and inoculation of periodontal pathogens.
3. Construction of a periodontitis+T. gondii-infected rat composite model: The T. gondii infection model is constructed first, then the method for constructing the periodontitis model is performed on the rats already infected with T. gondii to ultimately construct the periodontitis rat model.
4. Experimental design: Experimental animals are randomly divided into multiple groups.
(1) Blank control group: No silk suture ligature, no periodontal pathogens, and no T. gondii infection.
(2) Periodontitis group: Silk suture ligature of the first maxillary molar+infection with periodontal pathogens.
(3) T. gondii group: T. gondii infection followed by silk suture ligature of the first maxillary molar.
(4) Periodontitis+T. gondii group: T. gondii infection followed by silk suture ligature of the first maxillary molar+infection with periodontal pathogens.

Compared with the related art, beneficial effects of the disclosure are specifically as follows.
1) The disclosure uses co-infection of rats with P. gingivalis, T. denticola, and T. gondii to successfully obtain the periodontitis rat model in just 7 days. The constructed periodontitis rat model exhibits gingival papilla edema, gingival margin not adhering to the tooth surface, soft texture, and all show bleeding and periodontal pockets. As the severity increases, tooth mobility and even root exposure occur, which are consistent with the symptoms, signs, and pathological processes of human periodontitis, reflecting the systemic nature and severity of periodontitis. In contrast, the related art typically requires about one month to achieve a similar degree of the periodontitis model. Thus, the construction method of the disclosure greatly reduces the modeling time.
2) The disclosure not only rapidly obtains the periodontitis rat model by screening different combinations of common periodontitis pathogens but also has the advantages of high success rate, good controllability, and excellent stability, which can be used to study the development mechanisms and treatment and prevention processes of periodontitis.

### BRIEF DESCRIPTION OF DRAWINGS

FIG. 1 illustrates states of rats on the 5th day after intraperitoneal inoculation of T. gondii tachyzoites (upper right is a: T. gondii preservation rat; lower left is b: control group rat).

FIG. 2 illustrates a rat immobilized on a restraining board.

FIG. 3 illustrates periodontal silk suture ligature.

FIG. 4 illustrates intraoral inoculation with periodontal pathogens.

FIG. 5 illustrates oral gavage of T. gondii cysts in the rat.

FIG. 6 illustrates periodontal condition of the rat before ligation.

FIG. 7 illustrates periodontal condition of the rat in a blank control group.

FIG. 8 illustrates periodontal condition of the rat in a periodontitis group after ligation.

FIG. 9 illustrates periodontal condition of the rat in a periodontitis+T. gondii group after ligation.

FIG. 10 illustrates that T. gondii cysts are visible in a brain tissue homogenate of SD rats in the T. gondii group.

FIG. 11 illustrates histopathological changes in periodontal tissues of the rats in the blank control group (A), the periodontitis+T. gondii group (B), the periodontitis group (C), and the T. gondii group (D).

FIG. 12 illustrates micro-computed tomography (micro-CT) scan results of the periodontal tissues of the rats in the blank control group (A), the periodontitis+T. gondii group (B), the periodontitis group (C), and the T. gondii group (D).

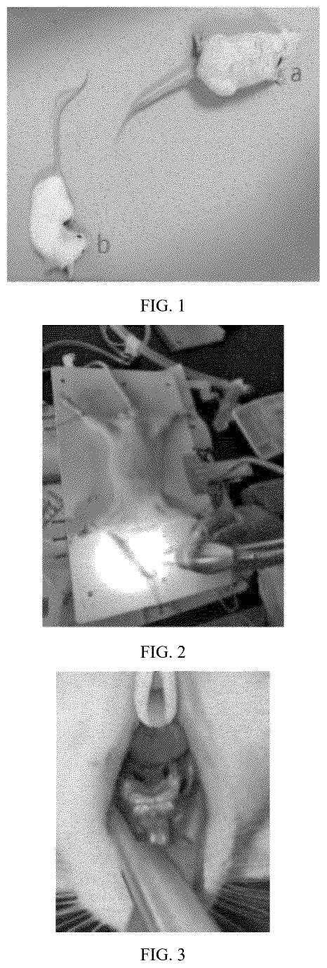

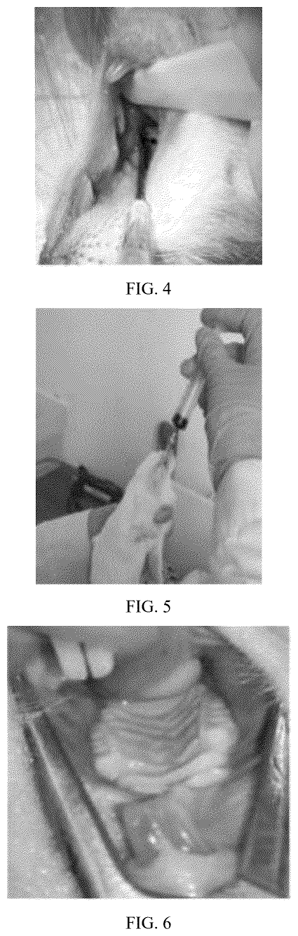

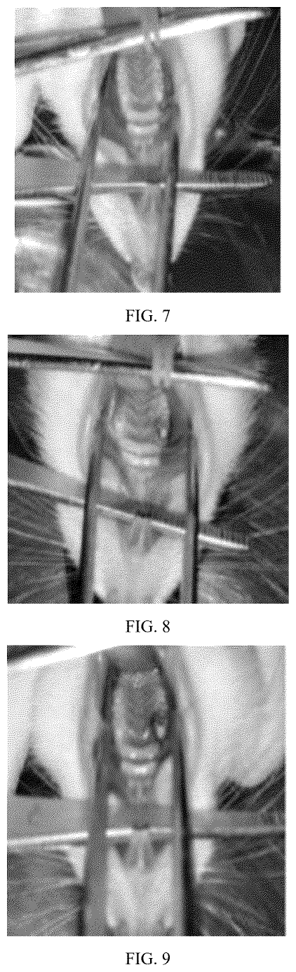

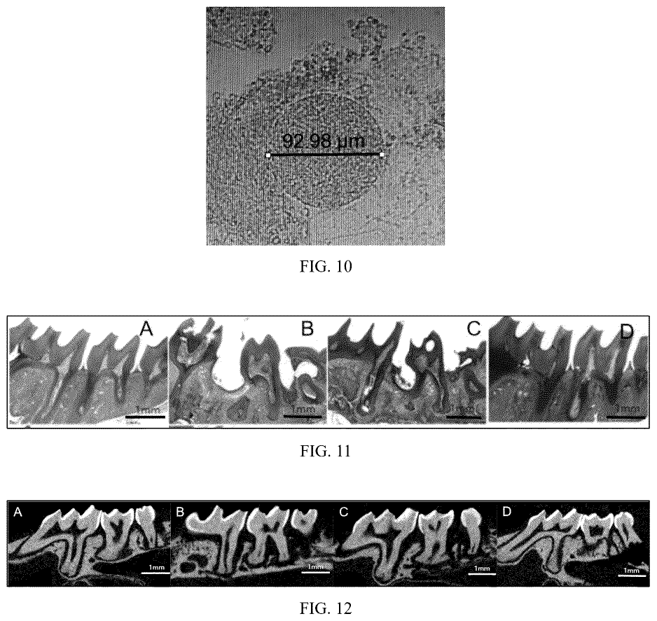

### DETAILED DESCRIPTION OF EMBODIMENTS

To facilitate a better understanding of the disclosure, specific embodiments are described below to provide further explanation. Terms used in the embodiments are for the description of particular implementations only and do not constitute a limitation on the scope of protection of the disclosure.

Unless otherwise specified, experimental methods not specifically conditioned in the following embodiments are generally carried out under conventional conditions or as recommended by the manufacturer. Materials, reagents, etc. used, unless otherwise specified, are all commercially available. Unless otherwise stated, substances mentioned in the disclosure are calculated by mass percentage and mass parts.

P. gingivalis: Strain No.: ATCC 33277, provided by the Laboratory of the School of Stomatology, Guangzhou Medical University, China.

T. denticola: Strain No.: DSM 14222, provided by the Laboratory of the School of Stomatology, Guangzhou Medical University, China.

T. gondii: Strain No.: T. gondii Me49, provided by the Parasitology Laboratory, College of Veterinary Medicine, South China Agricultural University, China.

Sprague-Dawley (SD) rats: purchased from the Laboratory Animal Center of Southern Medical University, China, Production/Experimental Animal License No.: SCXK (Yue) 2021-0041.

This experiment is conducted in the Animal Laboratory of the College of Veterinary Medicine, South China Agricultural University, China, Experimental Animal Use License Number: SYXK (Yue) 2024-0136.

The disclosure provides a method for constructing a rat model infected with P. gingivalis and T. denticola in synergy with T. gondii. The specific implementation steps of this method are described in detail below.

### 1. Preparation of Experimental Materials

1.1 Experimental animals: Specific pathogen-free (SPF)-grade 4-week-old male SD rats with a body weight of 200+20 g are selected. The animals are housed in a SPF central clean animal room at an ambient temperature of 22±2° C. and humidity of 50%+10%.
1.2 Bacteria: Standard strain of P. gingivalis ATCC 33277 and standard strain of T. denticola DSM 14222.
1.3 T. gondii: T. gondii Me49 strain.
1.4 Main materials and reagents: Brain heart infusion (BHI) medium, hemin, paraformaldehyde, vitamin K1.
1.5 Main experimental instruments: including anaerobic incubator, automatic dehydrator, paraffin embedding machine, paraffin tissue microtome, etc.
1.6 Preparation of main reagents: BHI solid medium and liquid medium are prepared according to reagent instructions.

### 2. Experimental Methods

2.1 Periodontitis group: Periodontitis is induced by ligating the first maxillary molar of rats and applying P. gingivalis and T. denticola.

T. gondii group: Rats are infected by oral gavage of T. gondii cysts.

Periodontitis+T. gondii group: The above two methods are combined to simultaneously induce periodontitis and T. gondii infection.

Blank control group: Physiological saline is used in all infection steps, and the rats are reared under the same environment and conditions.

Comparative group 1: F. nucleatum is used to replace T. denticola for infection, with all other conditions being the same as the periodontitis+T. gondii group.

Comparative group 2: T. forsythia is used to replace T. denticola for infection, with all other conditions being the same as the periodontitis+T. gondii group.

Comparative groups 3-4: Periodontitis is induced by ligating the first maxillary molar of rats and applying P. gingivalis and T. denticola, differing from the periodontitis+T. gondii group in that the usage ratio of P. gingivalis and T. denticola is adjusted.

F. nucleatum ATCC 2558 and T. forsythia ATCC 700198 are provided by the Oral Microbiology Research Laboratory, Guangzhou Medical University, China.
2.2 Sample collection and detection methods: including periodontal symptom examination, imaging examination, histopathological examination, etc.

### Embodiment 1 Periodontitis+T. gondii Group

### Construction of a Periodontitis Rat Model:

(1) Clean-grade SD rats are selected and subjected to 7 days of adaptive feeding.
(2) A Kunming rat chronically infected with T. gondii is euthanized, the whole brain is removed, homogenized, and diluted with physiological saline to a concentration of 100 brain cysts per milliliter (mL) to obtain an infection solution. After 7 days of adaptive feeding, each rat is administered 1 mL of the infection solution via oral gavage.
(3) The rats treated in step (3) are anesthetized by intraperitoneal injection of 1 mL of 1.5% sodium pentobarbital.
(4) After anesthesia, 3-0 silk suture ligatures are placed around a cervical region of first molars on both sides of an upper jaw of the anesthetized rat. Subsequently, a P. gingivalis bacterial suspension at a concentration of 1×10⁹ colony forming units per milliliter (CFU/mL) and T. denticola bacterial suspension at a concentration of 2×10⁹ CFU/mL are inoculated at a dose of 0.1 mL per tooth onto the ligature and into the gingival sulcus of the rat. The inoculation frequency is once daily for 7 consecutive days.
(5) The successful modeling is determined by measuring the periodontal symptoms and alveolar bone resorption distance of the rats treated in step (4). The alveolar bone resorption rate is ≥20%, and the periodontal probing depth is ≥1 millimeter (mm). Both conditions being met simultaneously indicate successful modeling.
(6) Oral, imaging, and histopathological examinations are performed on the rats treated in step (4).

### Embodiment 2 Periodontitis Group

### Construction of a Periodontitis Rat Model

(1) Clean-grade SD rats are selected and subjected to 7 days of adaptive feeding.
(2) Each rat is administered 1 mL of physiological saline via oral gavage.
(3) The rats treated in step (2) are anesthetized by intraperitoneal injection of 1 mL of 1.5% sodium pentobarbital.
(4) After anesthesia, 3-0 silk suture ligatures are placed around a cervical region of first molars on both sides of an upper jaw of the anesthetized rat. Subsequently, a P. gingivalis bacterial suspension at a concentration of 1×10⁹ CFU/mL and T. denticola bacterial suspension at a concentration of 2×10⁹ CFU/mL are inoculated at a dose of 0.1 mL per tooth onto the ligature and into the gingival sulcus of the rat. The inoculation frequency is once daily for 7 consecutive days.
(5) The successful modeling is determined by measuring the periodontal symptoms and alveolar bone resorption distance of the rats treated in step (4). The alveolar bone resorption rate is >20%, and the periodontal probing depth is ≥1 mm. Both conditions being met simultaneously indicate successful modeling.
(6) Oral, imaging, and histopathological examinations are performed on the rats treated in step (4).

### Embodiment 3: T. gondii Group

(1) Clean-grade SD rats are selected and subjected to 7 days of adaptive feeding.
(2) A Kunming rat chronically infected with T. gondii is euthanized, the whole brain is removed, homogenized, and diluted with physiological saline to a concentration of 100 brain cysts per mL to obtain an infection solution. After 7 days of adaptive feeding, each rat is administered 1 mL of the infection solution via oral gavage.
(3) The rats treated in step (2) are anesthetized by intraperitoneal injection of 1 mL of 1.5% sodium pentobarbital.
(4) After anesthesia, 3-0 silk suture ligatures are placed around a cervical region of first molars on both sides of an upper jaw of the anesthetized rat. Subsequently, physiological saline is inoculated at a dose of 0.1 mL per tooth onto the ligature and into the gingival sulcus of the rat. The inoculation frequency is once daily for 7 consecutive days;
(5) The successful modeling is determined by measuring the periodontal symptoms and alveolar bone resorption distance of the rats treated in step (4). The alveolar bone resorption rate is ≥20%, and the periodontal probing depth is ≥1 mm. Both conditions being met simultaneously indicate successful modeling.
(6) Oral, imaging, and histopathological examinations are performed on the rats treated in step (4).

### Embodiment 4: Blank Control Group

(1) Clean-grade SD rats are selected and subjected to 7 days of adaptive feeding.
(2) Each rat is administered 1 mL of physiological saline via oral gavage.
(3) The rats treated in step (2) are anesthetized by intraperitoneal injection of 1 mL of 1.5% sodium pentobarbital.
(4) After anesthesia, 3-0 silk suture ligatures are placed around a cervical region of first molars on both sides of an upper jaw of the anesthetized rat. Subsequently, physiological saline is inoculated at a dose of 0.1 mL per tooth onto the ligature and into the gingival sulcus of the rat. The inoculation frequency is once daily for 7 consecutive days.
(5) The successful modeling is determined by measuring the periodontal symptoms and alveolar bone resorption distance of the rats treated in step (4). The alveolar bone resorption rate is ≥20%, and the periodontal probing depth is ≥1 mm. Both conditions being met simultaneously indicate successful modeling.
(6) Oral, imaging, and histopathological examinations are performed on the rats treated in step (4).

The preparation methods of the comparative group 1 and the comparative group 2 are the same as the embodiment 1, differing from the embodiment 1 only in the respective specified bacterial species used in the comparative group 1 and the comparative group 2.

The preparation method of the comparative group 3 differs from the embodiment 1 in that in step (4), the concentration of the P. gingivalis bacterial suspension is 1.5×10⁹ CFU/mL, and the concentration of the T. denticola bacterial suspension is 1.5×10⁹ CFU/mL, with all other steps and parameters being identical.

The preparation method of the comparative group 4 differs from the embodiment 1 in that in step (4), the concentration of the P. gingivalis bacterial suspension is 2×10⁹ CFU/mL, and the concentration of the T. denticola bacterial suspension is 1×10⁹ CFU/mL, with all other steps and parameters being identical.

There are a total of eight test groups, with 20 rats in each group.

### 3. Sample Collection and Oral Examination, Imaging, and Histopathological Detection

Oral examination: Changes in gingival color and texture, probing depth, and tooth mobility are examined. One week after modeling, the free gingiva and gingival papillae of the sample rats are observed for color changes, whether they appear bright red or dark red, and whether the attached gingiva is involved; whether gingival tissue is swollen and the extent of swelling; changes in the stippling of the gingival surface; whether the gingival margin is thickened and adheres to the tooth surface; whether suppuration or erosion is present at the ligation site; and the texture of the gingiva, whether it lacks elasticity or is soft. Bleeding upon probing is checked, and the presence and depth of periodontal pockets are measured; and tooth mobility of the experimental tooth is also assessed.

The criteria for successful model validation by oral examination are as follows: the free gingiva and gingival papillae are bright red or dark red and involve the attached gingiva; the gingival tissue is swollen, the gingival margin is thickened and not attached to the tooth surface; suppuration or erosion is present at the ligation site; the gingiva is soft or lacks elasticity; bleeding upon probing is observed; the periodontal pockets are detectable; and tooth mobility is present.

Preparation of specimens for imaging and histopathological examination: For rats that meet the evaluation criteria for imaging and histopathological examination after step (5), maxillary bone samples and periodontal tissue structure specimens are collected. To preserve the structure of the first molar periodontal tissue to the greatest extent, cardiac perfusion fixation is used. The cardiac perfusion procedure is as follows: systemic anesthesia is induced by intraperitoneal injection of 1 mL of 1.5% pentobarbital sodium solution, and the rat's respiration and heartbeat are monitored continuously. The limbs and the incisors of the head are secured with rubber bands and fixed on a rat board (i.e., restraining board) in a supine position to begin cardiac perfusion fixation. After anesthesia, the chest wall is incised along the mid-axillary line at the level of the lower ribs, a 5 cm lateral incision is made, the liver and diaphragm are carefully separated with scissors, and the anterior chest wall is flipped upward to expose the heart. The ventricle is gently clamped and fixed with hemostatic forceps, then the blunt tip of a syringe needle is gently rotated and inserted approximately 1 mm into the ascending aorta at the apex of the heart. The right atrial appendage is cut open with a scissor. 50 mL of physiological saline is injected, when clear fluid flows out from the right atrial appendage and the rat's liver turns white, perfusion with 4% paraformaldehyde fixative (equal in volume to the rat's body weight) is continued. Subsequently, the maxillary bone is dissected and excised, periodontal tissues are collected, and stored in 4% paraformaldehyde.

Imaging examination: Specimens of the maxillary bone are subjected to micro-CT scanning and three-dimensional reconstruction, and alveolar bone resorption is measured.

Histopathological examination: Hematoxylin and eosin (HE) staining is performed on the periodontal tissue specimens to observe pathological morphological changes in the periodontal tissues. The HE staining procedure is carried out according to the instructions of the HE staining kit. The HE staining kit is purchased from Solarbio Science & Technology Co., Ltd., product code G1120.

$$Modeling success rate = ( number of successfully modeled rats / total number of rats in the group ) \times 100 \%$$

$$Mean alveolar bone resorption rate = ( sum of alveolar bone resorption rates for all rats in the group / total number of rats in the group ) \times 100 \%$$

$$Mean periodontal pocket probing depth = ( sum of periodontal pocket probing depths for all rats in the group / total number of rats in the group )$$

All results mentioned above are reported to two significant figures.

### 4. Experimental Results

TABLE 1
Detection of model rat samples

| | | Significance | | Significance | |
|---|---|---|---|---|---|
| | Mean | (compared with the | Mean | (compared with the | |
| | alveolar | blank control | periodontal | blank control | |
| | bone | group/the | pocket | group/the | Modeling |
| Experimental | resorption | periodontitis + | probing | periodontitis + | success |
| group | rate/% | T. gondii group) | depth/mm | T. gondii group) | rate/% |
| Periodontitis | 12.4 | */# | 0.72 | */# | 5 |
| group | | | | | |
| T. gondii | 8.5 | */# | 0.52 | */# | 0 |
| group | | | | | |
| Periodontitis + | 31.2 | ***/ | 1.65 | ***/ | 95 |
| T. gondii | | | | | |
| group | | | | | |
| Blank control | 3.2 | /# | 0.22 | /# | 0 |
| group | | | | | |
| Comparative | 21.5 | */# | 1.12 | */# | 50 |
| group 1 | | | | | |
| Comparative | 20.4 | */# | 1.04 | */# | 40 |
| group 2 | | | | | |
| Comparative | 23.5 | */# | | */# | 70 |
| group 3 | | | | | |
| Comparative | 24.2 | */# | | */# | 75 |
| group 4 | | | | | |

Note:
“*” indicates a significant difference between the experimental group and the blank control group;
“#” indicates a significant difference between the experimental group and the periodontitis + T. gondii group;
“*” represents p < 0.05,
“**” represents p < 0.01,
“***” represents p < 0.001;
“#” represents p < 0.05;
“ns” indicates no significant difference compared to the blank control group or the periodontitis + T. gondii group.

The above experimental results indicate that the method for constructing the periodontitis rat model through co-infection with P. gingivalis, T. denticola, and T. gondii achieves a modeling success rate of over 95% within 7 days, demonstrating a short modeling time and high success rate. In addition, oral, imaging, and histopathological examinations are performed on the 19 rats in which modeling is successful. Oral examination results show that all 19 successfully modeled rats in the periodontitis+T. gondii group exhibit dark red gingival papillae, swollen gingival tissue, erosion at the ligation site, soft gingival texture, and bleeding upon probing.

HE staining observations show that in the blank control group (portion A in FIG. 11), the junctional epithelium shows no attachment loss and is located at the cementoenamel junction, with intact tooth structure. In the periodontitis group, slight epithelial proliferation into the connective tissue is observed, with minor resorption of the alveolar crest and alveolar bone proper, and few osteoclasts are present in the alveolar bone. However, in the periodontitis group (portion C in FIG. 11), loss of junctional epithelial attachment and alveolar bone destruction are observed, with osteoclasts visible, and their number is increased compared to the T. gondii group (portion D in FIG. 11). In the periodontitis+T. gondii group (portion B in FIG. 11), significant loss of junctional epithelial attachment and significant alveolar bone destruction are observed, with numerous osteoclasts present, and their number is significantly increased compared to both the periodontitis group (portion C in FIG. 11) and the T. gondii group (portion D in FIG. 11).

Micro-CT results show that, compared to the blank control group (portion A in FIG. 12), the periodontitis group (portion C in FIG. 12), and the T. gondii group (portion D in FIG. 12), the height, integrity, and fullness of the alveolar bone are significantly reduced in the periodontitis+T. gondii group (portion B in FIG. 12).

Specifically, comparison of the results from the rat model of the periodontitis+T. gondii group and the periodontitis group and T. gondii group demonstrates as follows.
(1) The periodontitis rat model cannot be effectively constructed within 7 days by infection with periodontal pathogens alone or T. gondii alone. Only one out of 20 rats in the periodontitis group shows obvious periodontitis symptoms, specifically gingival redness, slight bleeding upon probing, and visibly swollen gingival tissue, while no obvious symptoms are observed in the other 19 rats in this group or in the T. gondii group.
(2) Infection with T. gondii exacerbates the severity of periodontitis, manifested as worsening clinical symptoms and increased alveolar bone resorption in imaging. In the rat model, this exacerbating effect is statistically significant (P<0.001).

Comparisons between the rat model of the periodontitis+T. gondii group and the comparative groups 1-2 demonstrates as follows. The infection effects of different periodontal pathogens vary significantly. The combination of P. gingivalis and T. denticola produces a significantly better outcome than the combination of P. gingivalis with F. nucleatum or the combination of P. gingivalis with T. forsythia, indicating that co-infection with P. gingivalis and T. denticola is more suitable for synergistic rapid construction of the periodontitis rat model with T. gondii infection, and significantly affects modeling success rate.

Comparisons between the rat model of the periodontitis+T. gondii group and the comparative groups 3-4 demonstrates as follows. Different ratios of the same bacterial strains also influence modeling outcomes to some extent. The ratio of P. gingivalis to T. denticola used in the disclosure is 1:2, which provides optimal infection efficiency, further enhancing the modeling success rate.

Conclusion: The periodontitis rat model is successfully constructed in this disclosure. According to the experimental results, a certain synergistic effect is observed between the T. gondii infection and co-infection with P. gingivalis and T. denticola. Moreover, the model is simple to construct, requires a short modeling time, exhibits high reproducibility, and can be produced in batches. Through this model, the pathological process of human P. gingivalis+T. denticola infection accompanied by T. gondii infection can be effectively simulated, providing a powerful tool for studying the influence and mechanisms of T. gondii infection on the progression of periodontitis.

The above is only the implementation of the disclosure, and its description is more specific and detailed, but it should not be interpreted as limiting the patent scope of the disclosure. It should be noted that, for those skilled in the art, various modifications and changes can still be made without departing from the inventive concept of the disclosure, and all such modifications and changes fall within the protection scope of the disclosure.

What is claimed is:

1. A method for constructing a periodontitis rat model, comprising the following steps:
(1) selecting clean-grade Sprague-Dawley (SD) rats and performing 7 days of adaptive feeding before infection on the SD rats;
(2) euthanizing a Kunming rat chronically infected with Toxoplasma gondii, extracting a whole brain, grinding and homogenizing the whole brain to a mixture, and diluting the mixture with physiological saline to a concentration of 100 brain cysts per milliliter (mL) to obtain an infection solution; administering 1 mL of the infection solution to each rat via oral gavage after 7 days of adaptive feeding to obtain treated rats;
(3) anesthetizing the treated rats in step (2) with 1 mL of 1.5% sodium pentobarbital via intraperitoneal injection to obtain anesthetized rats;
(4) using 3-0 silk suture to ligate a cervical region of first molars on both sides of an upper jaw of the anesthetized rats, then inoculating 0.1 mL per tooth with Porphyromonas gingivalis bacterial suspension at a concentration of 1×10⁹ colony forming units per milliliter (CFU/mL) and Treponema denticola bacterial suspension at a concentration of 2×10⁹ CFU/mL, both administered intraorally onto the silk suture and within gingival sulcus of the anesthetized rats; performing inoculation once daily for 7 days consecutively; and
(5) after 7 days, measuring and observing alveolar bone and periodontal pocket conditions of the rats treated in step (4) after the inoculation of 7 days to determine whether modeling is successful; and
wherein in step (5), the alveolar bone and periodontal pocket conditions are defined as alveolar bone resorption rate greater than or equal to 20% and periodontal probing depth greater than or equal to 1 millimeter (mm), with both conditions being met simultaneously indicating successful construction of the periodontitis rat model.

2. The method for constructing the periodontitis rat model as claimed in claim 1, wherein a rat body weight is in a range of 180 grams (g) to 220 g.

3. The method for constructing the periodontitis rat model as claimed in claim 1, further comprising:
performing oral, imaging, and histopathological examinations on the rats treated in step (4) after successful modeling.

4. The method for constructing the periodontitis rat model as claimed in claim 3, wherein steps for the oral, imaging, and histopathological examinations are as follows:
A1: administering 1 mL of 1.5% sodium pentobarbital solution via intraperitoneal injection to induce general anesthesia;
A2: securing incisors with rubber bands around limbs and head of the rats treated in step A1, fixing the rats treated in step A1 on a board in a supine position;
A3: after anesthesia, making an incision along an anterior midline of a thoracic wall below ribs, performing a lateral cut of 5-6 centimeters (cm), separating liver and peritoneum, opening thorax, and exposing heart;
A4: fixing a ventricle, then inserting a needle tip of a syringe at an apex into an ascending aorta for a depth of 1 mm;
A5: cutting open a right atrial appendage with a scissor, injecting 50 mL of physiological saline, allowing a fluid to flow out from the right atrial appendage until a liver of the rat turns pale, continuing perfusion with 4% paraformaldehyde solution at a body weight, terminating the perfusion upon completion, dissecting and harvesting a maxillary bone and periodontal tissues of the rat, and preserving the maxillary bone and the periodontal tissues in 4% paraformaldehyde to obtain a maxillary bone specimen and a periodontal tissue specimen;
A6: performing micro-computed tomography (micro-CT) scanning and three-dimensional reconstruction on the maxillary bone specimen; and
A7: staining the periodontal tissue specimen with hematoxylin and eosin (HE) to observe morphological changes in the periodontal tissues.

5. An application of the periodontitis rat model obtained by the method as claimed in claim 1 in study of mechanisms of periodontitis occurrence.
